(19) United States
(12) Patent Application Publication
(10) Pub. No.: US 2026/0076992 A1
(43) Pub. Date: Mar. 19, 2026

(54) COMPOSITIONS AND METHODS FOR TREATING VAGINAL ATROPHY

(71) Applicants: Janet Mubo AKANDE, Johns Creek, GA (US); Oreoluwa Olufunmilayo ADEDOYIN, Hoover, AL (US)

(72) Inventors: Janet Mubo AKANDE, Johns Creek, GA (US); Oreoluwa Olufunmilayo ADEDOYIN, Hoover, AL (US)

(21) Appl. No.: 19/109,039

(22) PCT Filed: Sep. 6, 2023
(86) PCT No.: PCT/US2023/032117

§ 371 Date: Mar. 5, 2025

Related U.S. Application Data
(60) Provisional application No. 63/374,640, filed on Sep. 6, 2022.

(51) Int. Cl. A61K 31/732; A61K 9/00; A61K 9/02; A61K 9/06; A61K 9/08; A61K 31/728; A61K 38/39; A61K 47/10; A61K 47/12; A61P 15/02

(52) U.S. Cl. CPC A61K 31/732; A61K 9/0034; A61K 9/02; A61K 9/06; A61K 9/08; A61K 31/728; A61K 38/39; A61K 47/10; A61K 47/12; A61P 15/02

(57) ABSTRACT

Compositions and methods for treating one or more symptoms associated with vaginal atrophy or discomfort are disclosed. The compositions include an effective amount of one or more of the active agents selected from pectin (P), hyaluronic acid (HA), and collagen (Cg), in a pharmaceutically acceptable carrier, for vaginal delivery. The disclosed compositions are useful in treating one or more symptoms associated with vaginal atrophy. Example subjects include postmenopausal women or premenopausal women who experience these symptoms as a result of breastfeeding, intensive vaginal douching, cigarette smoking, autoimmune conditions such as Sjogren's syndrome, pathologic conditions such as diabetes, premature ovarian insufficiency, bilateral oophorectomy, ovarian failure due to radiation or arterial embolization, hypothalamic-pituitary disorders. Additionally, the disclosed compositions are useful in generally alleviating vaginal discomfort such as pH imbalance, vaginal dryness, burning, itching, vaginal discomfort, pain and burning when urinating, dyspareunia (painful sex), and spotting during intercourse.

### CROSS-REFERENCE TO RELATED APPLICATIONS

This application claims benefit of U.S. Provisional Application No. 63/374,640 filed Sep. 6, 2023, which is incorporated herein by reference in its entirety.

### FIELD OF THE INVENTION

The invention is generally in the field of compositions and methods for treating one or mor symptoms associated with vaginal atrophy.

### BACKGROUND OF THE INVENTION

Vaginal atrophy (VA) is characterized by a thinning or degradation of vaginal epithelium due to loss of circulating estrogen. It is common in post-menopausal women or women of any age who have decreased estrogen production due to use of bilateral oophorectomy, ovarian failure, use of antiestrogenic medications etc. Symptoms of VA include but are not limited to vaginal dryness, burning, itching, bleeding, spotting etc. The epidemiology of VA is generally as follows: before menopause: 15% of women; after menopause: 40-57% of women. However, VA is generally under-reported since 70% of women do not report symptoms to their provider and less than 25% do not receive care.

Current treatment options are as follows: first line treatment includes non-hormonal use of moisturizers and lubricants—generally available for self-management. The 2^{nd} line treatment involves use of estrogen and non-estrogen-based therapies—requires clinician prescription. Other management options include: alternative and complementary medicines; these have limited data to support efficacy. In addition, energy based medical device such as laser or radiofrequency devices which are noninvasive procedures that are used to induce collagen production and vascularization of the vaginal epithelium in order to restore elasticity of the vaginal mucosa may also be used.

There are several notable limitations to current treatments. In practice, while most clinicians find estrogen therapy to be more effective than non-hormonal treatments, the concern for systemic absorption of local estrogen is associated with the risks of estrogen-based therapy which includes thrombosis makes hormonal-based therapy less appealing for patients. Corrective minimally invasive procedures such as radiofrequency or laser-based devices have shown some effectiveness in improving symptoms of vaginal atrophy. However, these devices have not been cleared by the FDA for use in urogynecology and are not generally recommended due to the lack of randomized clinical trials to demonstrate long term safety and efficacy. In addition, these procedures while performed in some gynecological offices are generally not covered by the insurance companies.

Therefore, there remains a need for novel therapies for treatment of VA.

It is therefore an object of the present invention to provide compositions for treatment of VA.

It is also an object of the present invention to provide methods of treating VA.

### SUMMARY OF THE INVENTION

Compositions and methods for treating one or more symptoms associated with vaginal atrophy are disclosed. The compositions include an effective amount of an active agent pectin (P) with an optional second active agent, hyaluronic acid (HA), active agent collagen (Cg), or a combination of HA and Cg, in a pharmaceutically acceptable carrier, for vaginal delivery. In one embodiment, the compositions include an effective amount of P in combination with HA. In another embodiment the compositions include an effective amount of P in combination with Cg. In still another embodiment, the compositions include a combination of P, HA and Cg.

The compositions are effective to increase gene expression of COL1A1 and/or COL3A1 in human epithelial vaginal cells, following administration. In one embodiment, the compositions are effective to increase gene expression of COL1A1 and COL3A1, when compared to gene expression seen with untreated controls, or in combination with HA and Cg. The compositions are effective to stimulate endogenous synthesis of collagen protein in human vaginal epithelial cells as evidenced by the disclosure in FIG. 1.

The disclosed compositions are useful in treating one or more symptoms associated with vaginal atrophy, and include, but are not limited to vaginal pH imbalance, vaginal dryness, burning, itching, vaginal discomfort, pain and burning when urinating, dyspareunia (painful sex), and spotting during intercourse. The compositions are administered to a subject in need thereof, such as postmenopausal women or women of any age who experience a decrease in estrogenic stimulation of the urogenital tissues resulting in changes to the anatomy and physiology of the genitourinary system, premenopausal women who experience these symptoms as a result of breastfeeding, intensive vaginal douching, cigarette smoking, autoimmune conditions such as Sjogren's syndrome, pathologic conditions such as diabetes, premature ovarian insufficiency (e.g., due to premature aging), bilateral oophorectomy, ovarian failure due to radiation or arterial embolization, hypothalamic-pituitary disorders. as well as medication classes such as antidepressants, antiestrogenic (e.g., to treat endometriosis and uterine fibroids), and anticholinergic medications.

In one aspect, the composition disclosed herein is for treating one or more symptoms associated with vaginal atrophy or discomfort. The composition has a pH between 3.5 and 4.5 and comprises an effective amount of an active agent pectin (P) with an optional second active agent selected from the group consisting of hyaluronic acid (HA), collagen (Cg), and a combination of HA and Cg, in a pharmaceutically acceptable carrier, for vaginal delivery.

In one embodiment, the composition disclosed herein comprises an effective amount of P only. In one embodiment, the composition disclosed herein comprises an effective amount of P in combination with HA. In one embodiment, the composition disclosed herein comprises an effective amount of P in combination with Cg. In one embodiment, the composition disclosed herein comprises an effective amount of P, HA, and Cg.

In one embodiment, the pharmaceutically acceptable carrier of the compositions disclosed herein comprises at least one of glycerin and phenoxyethanol. In one embodiment, the pharmaceutically acceptable carrier of the compositions disclosed herein comprises lactic acid. In one embodiment, the composition disclosed herein further comprises additives, for example, additives to provide delayed release.

In one embodiment, the composition disclosed herein has a consistency of a gel, a viscous liquid, or somewhere in-between the consistency of a gel and a viscous liquid. In one embodiment, the composition disclosed herein comprises by w/w %, 0.1-30% pectin, 0-15% Cg, and 0-10% HA. In one embodiment, the composition disclosed herein comprises by w/w %, 0-30% glycerin, 0-5% lactic acid, 0-1% phenoxyethanol, and 9-90% water.

In another aspect, the present disclosure is directed to a formulation comprising the composition disclosed herein, formulated into a gel, a vaginal suppository. In one embodiment, the formulation disclosed herein is encapsulated in a vaginal applicator. In one embodiment, the formulation disclosed herein is infused into a sheet of absorbent or porous material to form a tissue, a wipe, a towel, a towelette, or the like.

In a third aspect, the present disclosure is directed to a method of treating one or more symptoms associated with vaginal atrophy or discomfort that comprises the step of administering the composition or formulation disclosed herein, to a subject in need thereof. In one embodiment, the one or more symptoms to be treated are selected from the group consisting of pH imbalance, vaginal dryness, burning, itching, vaginal discomfort, pain and burning when urinating, dyspareunia (painful sex), and spotting during intercourse. In one embodiment, the subject is a postmenopausal women.

### BRIEF DESCRIPTION OF THE DRAWINGS

In addition to the common abbreviation of pectin to P, hyaluronic acid to HA, and active ingredient collagen to Cg, commonly detected collagen is abbreviated as COL or Col in the figures and throughout the present disclosure. Control is abbreviated as Ctrl in the figures. β-Actin is abbreviated as β-ACTN in the figures.

The drawing figures are not necessarily to scale and certain features may be shown exaggerated in scale or in a somewhat generalized or schematic form in the interest of clarity and conciseness. For more complete understanding of the features and advantages of the present disclosure, reference is now made to the detailed description along with the accompanying figures, wherein:

FIG. 1 is a bar graph showing the effect of active agent concentration on endogenous stimulation of collagen production in human vaginal epithelial cells.

FIG. 2 is a bar graph showing the effect of pectin on three healthy vaginal bacteria strains.

FIGS. 3A-3C are representative graphs showing a dose dependent increase in gene expression of Col1A1 after treatment with formulations HA+P and HA only at concentrations 0.15 mg/mL, 1.5 mg/mL, and 3 mg/mL, respectively. At each concentration, the treatment with HA+P formulation either showed a trend towards increase or resulted in significant increase in Col1A1 gene expression compared to either the untreated controls or with the HA only formulation.

FIGS. 3D-3F, similar to FIG. 3A3-C, show the effect of HA+P and HA only on Col3A1 gene expression in hVECs, at concentrations 0.15 mg/mL, 1.5 mg/mL, and 3 mg/mL, respectively. Treatment of hVECs with HA+P resulted in significant increase in Col3A1 gene expression levels especially at 1.5 mg/mL and 3 mg/mL concentrations in FIGS. 3E and 3F, respectively, compared to control and compared to HA only treatments.

FIGS. 4A-4C show the effect of formulation Cg+P on expression of Col1A1 at concentrations 0.15 mg/mL, 1.5 mg/mL, and 3 mg/mL, respectively.

FIGS. 4D-4F show the effect of formulation Cg+P on expression of Col3A1 at concentrations 0.15 mg/mL, 1.5 mg/mL, and 3 mg/mL, respectively.

FIGS. 5A-5C show treatment of hVECs with formulation Cg on Col1A1 gene expression at concentrations 0.15 mg/mL, 1.5 mg/mL, and 3 mg/mL, respectively.

FIGS. 5D-5E show the effect of formulation Cg on Col3A1 gene expression, at concentrations 0.15 mg/mL and 3 mg/mL, respectively.

FIGS. 6A-6C show the effect on Col1A1 gene expression in hVECs treated with either formulation Cg or HA at different concentrations over time, at concentrations 0.15 mg/mL, 1.5 mg/mL, and 3 mg/mL, respectively.

FIG. 7 shows the effect of pectin in stimulating Cg+HA based Col1A1 induction. In the absence of pectin, Cg+HA formulation does not increase Col1A1 gene expression Analyses: 2-way ANOVA results and Mixed Effects analysis with Sidak's multiple comparisons test; p<0.05. Number of * denotes degree of significance.

### DETAILED DESCRIPTION OF THE INVENTION

### I. Definitions

A “cream” is a viscous liquid or semi-solid emulsion of either the “oil-in-water” or “water-in-oil type”.

An “emulsion” is a composition containing a mixture of non-miscible components homogenously blended together.

“Gel” as used herein is a colloid in which the dispersed phase has combined with the continuous phase to produce a semisolid material, such as jelly.

A “lotion” is a low- to medium-viscosity liquid formulation.

“Oil” as used herein refers to a composition containing at least 95% wt of a lipophilic substance. Examples of lipophilic substances include but are not limited to naturally occurring and synthetic oils, fats, fatty acids, lecithins, triglycerides and combinations thereof.

An “ointment” is a semisolid preparation containing an ointment base and optionally one or more active agents.

“Parenteral administration”, as used herein, means administration by any method other than through the digestive tract or non-invasive topical or regional routes.

“Patient” or “subject” to be treated as used herein refers to either a human or non-human animal.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “pudendum” refers to the external genitalia and surrounding regions of the body, including the interlabial folds, the clitoral region, the perineum, the perianal region, the vulvar and perivulvar regions, and the intergluteal folds.

“Therapeutically effective” or “effective amount” as used herein means that the amount of the composition used is of sufficient quantity to ameliorate one or more causes or symptoms of a disease or disorder. Such amelioration only requires a reduction or alteration, not necessarily elimination. As used herein, the terms “therapeutically effective amount” “therapeutic amount” and “pharmaceutically effective amount” are synonymous. One of skill in the art can readily determine the proper therapeutic amount.

### II. Compositions

Compositions useful for treating vaginal atrophy are disclosed, which include as active agents, including at least one of pectin (P), hyaluronic acid (HA), and collagen (Cg). The compositions are formulated for administration directly into the vaginal, in some embodiments using a convenient transvaginal application that deposits a very small volume of drug at the desired site for delivery. The compounds described herein are formulated for vaginal administration, which can be intravaginally, or topically deposited on the vaginal pudendum.

### Active Agents

### Pectin

Pectin is a complex mixture of polysaccharides that makes up about one third of the cell wall dry substance of higher plants. Its main component is galacturonic acid, a sugar acid derived from galactose. Pectins, also known as pectic polysaccharides, are rich in galacturonic acid. Several distinct polysaccharides have been identified and characterized within the pectic group. Homogalacturonans are linear chains of α-(1-4)-linked D-galacturonic acid. Substituted galacturonans are characterized by the presence of saccharide appendant residues (such as D-xylose or D-apiose in the respective cases of xylogalacturonan and apiogalacturonan) branching from a backbone of D-galacturonic acid residues. Rhamnogalacturonan I pectins (RG-I) contain a backbone of the repeating disaccharide: 4)-α-D-galacturonic acid-(1,2)-α-L-rhamnose-(1. From many of the rhamnose residues, sidechains of various neutral sugars branch off. The neutral sugars are mainly D-galactose, L-arabinose and D-xylose, with the types and proportions of neutral sugars varying with the origin of pectin. Another structural type of pectin is rhamnogalacturonan II (RG-II), which is a less frequent, complex, highly branched polysaccharide. Isolated pectin has a molecular weight of typically 60,000-130,000 g/mol, varying with origin and extraction conditions. commercial pectins are almost exclusively derived from citrus peel or apple pomace, both by-products from juice (or cider) manufacturing. Apple pomace contains 10-15% of pectin on a dry matter basis. Citrus peel contains of 20-30%. Methods of extracting pectins from citrus fruit such as orange or lemon peels are known in the art and commercially available. Specific type of pectin is a blend of low methoxyl and high methoxyl pectin such as those disclosed in Tas, et al., Coatings 2021, 11, 922.

Pectin used in the formulation disclosed herein can be of low methoxyl pectin, high methoxyl pectin, or a combination thereof, at a concentration range between 0.1 and 30% of the formulation, for example, between 1 and 20%, such as between 1-2%, 2-3%, 3-4%, 4-5%, 5-6%, 6-7%, 7-8%, 8-9%, 9-10%, 10-12%, 12-14%, 14-16%, 16-18%, or 18-20% etc. of the formulation.

### Hyaluronic Acid

Hyaluronic acid (HA) is a natural and linear polymer composed of repeating disaccharide units of β-1,3-N-acetyl glucosamine and β-1,4-glucuronic acid with a molecular weight up to 6 million Daltons. With excellent viscoelasticity, high moisture retention capacity, and high biocompatibility, HA finds a wide range of applications in medicine, cosmetics, and nutraceuticals. HA is a humectant i.e., a substance that retains moisture, and it is capable of binding over one thousand times its weight in water. This substance is naturally found in many areas of the human body, including the skin, eyes, and synovial fluid of the joints. There are two main sources of Hyaluronic Acid: plant origins and animal origins. Plant-based Hyaluronic Acid is extracted from microbial fermentation i.e., a bacterial strain naturally contains Hyaluronic Acid and is then fermented to yield the desired molecular weights ideal for skin care purposes (Lui, et al., Microb Cewll Fact., 10:99, 2011). Animal-based Hyaluronic Acid utilizes the combs of roosters—this is the red flesh at the top of a rooster's head. Like humans, other animals also produce Hyaluronic Acid in their bodies, and the rooster's comb is considered one of the best animal sources. Traditionally HA was extracted from rooster combs, and now it is mainly produced via microbial fermentation with lower production costs and less environmental pollution HA has been successfully produced on an industrial scale with Streptococcus sp. as the main producer.

HA can be used at a concentration range between about 0.1 and 10% of the formulation, for example, between 1 and 10%, such as 1-2%, 2-3%, 3-4%, 4-5%, 5-6%, 6-7%, 7-8%, 8-9%, 9-10% of the formulation. In one embodiment, the HA should have a molecular weight from about 1 KDa to 3 MDa.

### Collagen

Collagen is a principal protein of connective tissue. The main medical types of Collagen are I-V which are present in different body parts Type I is found in bones, tendons, ligaments and skin; Type II is in cartilage and the eyes; Type III comes from the lungs, liver and arteries Type IV is present in the kidneys; and Type V is found on the surface of cells, hair and placenta. There are at least 29 distinct kinds of collagen known to science. They are grouped into three categories based on their ability to generate fibrils. They are referred to as “fibril-forming colloids” because they produce banded fibrils and are found in the collagen types I through VIII. This group of collagens contains kinds IX, XII, XIV, and potentially even IX, XVI, XV, XVI, XVIII, & XVII, and types XVI, XVII, XVII, XXVI, & XXVII as well. Types IV, VIII, and X of network-forming collagens, types VI and VII of beaded collagens, types VI and VII of anchoring fibrils, and invertebrate cuticle collagens, comprise the third category of non-fibrillar collagens. They produce sheets of protein membranes around tissues and organisms. Deterioration of this protein causes wrinkles as we get older because of its role in the skin's strength and flexibility (Reviewed in Shenoy M, Abdul N, Qamar Z, et al. (May 9, 2022) Collagen Structure, Synthesis, and Its Applications: A Systematic Review. Cureus 14(5): e24856. doi:10.7759/cureus.24856). Collagens useful in the disclosed compositions include non-hydrolyzed and hydrolyzed collagen (Types I, II, III or IV). For example, type I and III from a fermentation-derived collagen such as those describe ed in Kerkhof et al., Int Urogynecol J. Pelvic Floor Dysfunct 2009; 20 (4); 461-474.

Collagen can be used at a concentration range between about 0.1 and 15% of the formulation, for example, between 1 and 10%, such as 1-2%, 2-3%, 3-4%, 4-5%, 5-6%, 6-7%, 7-8%, 8-9%, 9-10%, 10-11%, 11-12%, 12-13%, 13-14%, 14-15% etc. of the formulation.

The compositions can additionally include prebiotic chosen among gluco-oligosaccharides (GOS), fructo-oligosaccharides (FOS), galacto-oligosaccharides and mixtures thereof; and a plant extract containing isoflavones chosen among soybean (Glycine max) extracts, clover (Trifolium sp.).

The disclosed compositions and formulations in some respects, do not include growth hormone, ICE deep sea mineral crystal ions, glycerol, perilla oil, traditional Chinese medicine extract, yeast beta-glucan, inulin, plant-derived fructan and fructose oligomer, Lactic acid as active ingredient, lactobacillus, an aloe extract, a mint herb extract, sodium pyrrolidonecarboxylate, etc.

### Formulations

The compounds described herein can be formulated for intravaginal, or vaginal topical administration to the pudendum. Useful dosage forms, include, but are not limited to foams, creams, ointments, vaginal pessary/suppositories, tablets, sprays, gels including mucoadhesive gels, hydrogels and hydrogels from hydrocolloids, applied in some embodiments using a suitable applicator for intravaginal delivery, and for topical vaginal delivery, using devices such as feminine wipes, panty liners impregnated with effective amounts of the active agents. Intravaginal drug delivery methods and formulations are known in the art (Sahoo, et al., AJADD1[1][2013]043-055; Bakke, et al. Design aspects of vaginal applicators that influence acceptance among target users. Sci Rep 11, 9802 (2021)).

The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

The active agents described herein can be formulated for controlled release including immediate release, delayed release, extended release, pulsatile release, and combinations thereof.

In some embodiments, the one or more active agents and optional one or more additional active agents are dispersed in a matrix material, which gels or emulsifies upon contact with an aqueous medium, such as physiological fluids.

In the case of gels/hydrogels, the matrix swells entrapping the active agents, which are released slowly over time by diffusion and/or degradation of the matrix material. Such matrices can be formulated as tablets or as fill materials for hard and soft capsules as well as films.

In still another embodiment, the one or more compounds, and optional one or more additional active agents are formulated into a sold dosage form, such as a tablet or capsule, and the solid dosage form is coated with one or more controlled release coatings, such as a delayed release coatings or extended-release coatings. The coating or coatings may also contain the compounds and/or additional active agents.

An example formulation with excipients is shown in Table 1 below, indicating concentration ranges for active agents.

TABLE 1
Composition of an example formulation

| | Concentration | Molecular |
|---|---|---|
| Component | range (% w/w) | specificity |
| Pectin (P, | 0.1%-30%, such as | high methoxyl pectin |
| active agent) | 0.1-5%, 5-10%, | sourced from citrus |
| | 10-15%, 15-20%, | peel, having a MW of |
| | 20-25%, or 25-30% | 80-110 KDa. |
| Collagen (Cg, | 0%-15%, such as 0-2%, | Non-hydrolyzed and |
| active agent) | 2-4%, 4-6%, 6-8%, | hydrolyzed collagen |
| | 8-10%, 10-12%, or | (Types I, II, III or |
| | 12-15%. | IV), MW of about 5 kDa |
| Hyaluronic | 0%-10%, such as 0-2%, | Molecular weight range |
| acid (HA, | 2-4%, 4-6%, 6-8%, or | 1 KDa to 3 MDa |
| active agent) | 8-10%. | |
| Glycerin | 0%-30%, such as 0-5%, | |
| | 5-10%, 10-15%, 15-20%, | |
| | 20-25%, or 25-30% | |
| Lactic acid | 0%-5%, such as 0.01-1%, | Acting as pH adjuster, |
| | 1-2%, 2-3%, 3-4%, or | alternative embodiments |
| | 4-5%. | include other commonly |
| | | used pH adjusters. |
| Phenoxyethanol | 0%-1%, such as 0-0.2%, | Acting as preservatives, |
| | 0.2-0.4%, 0.4-0.6%, | alternative embodiments |
| | 0.6-0.8%, or 0.8-1% | include potassium |
| | | sorbate or sodium |
| | | benzoate” |
| Water | 9%-90%, such as 9-15%, | |
| | 15-20%, 20-25%, 25-30%, | |
| | 30-35%, 35-40%, 40-45%, | |
| | 45-50%, 50-55%, 55-60, | |
| | 60-70%, 70-80%, or | |
| | 80-90%. | |

### (a). Emulsions

An emulsion is a preparation of one liquid distributed in small globules throughout the body of a second liquid. In particular embodiments, the non-miscible components of the emulsion include a lipophilic component and an aqueous component. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Excipients include surfactants, especially non-ionic surfactants; emulsifying agents, for example emulsifying waxes; and liquid non-volatile non-aqueous materials, for example glycols such as propylene glycol. Example excipients are shown in Table 1. The oil phase may contain other oily pharmaceutically approved excipients. For example, materials such as hydroxylated castor oil or sesame oil may be used in the oil phase as surfactants or emulsifiers.

The oil phase may consist at least in part of a propellant, such as an hydrofluoroalkane (HFA) propellant. Either or both of the oil phase and the aqueous phase may contain one or more surfactants, emulsifiers, emulsion stabilizers, buffers, and other excipients. Excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

### (b). Lotions

A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

### (c). Creams

Creams may contain emulsifying agents and/or other stabilizing agents. In one embodiment, the formulation is in the form of a cream having a viscosity of greater than 1000 centistokes, typically in the range of 20,000-50,000 centistokes. Compared to ointments, creams are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%. Creams for vaginal delivery are known. WO 98/11888 discloses formulations including creams and gels, for intravaginal delivery of anti-incontinent agents.

### (d). Ointments

Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

### (e). Gels

Gels have been used for vaginal delivery of many active agents, which include several currently marketed gels (reviewed in Neves, Int. J. Pharmaceuticals, 318:1-14 (2006); See also, Barnhart, et al., Contraception, 72:65-70 (2005)). Gels are semisolid systems containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

In one embodiment, the gel formulation closed herein comprises pectin as the only active ingredient at a concentration of 0.2%-3% w/w, for example, between 0.2% to 0.5%, 0.5% to 1%, 1% to 1.5%, 1.5% to 2%, or 2.5% to 3% w/w. In one embodiment, the pectin formulation is pre-loaded as a gel in an applicator. In one embodiment, the pectin formulation in gel format in an applicator is used for to balance vaginal pH to it is desired 3.5-4.5 range.

In one embodiment, the gel formulation disclosed herein comprises both pectin (P) and hyaluronic acid (HA) as the active ingredients, with the pectin at a concentration of 0.2%-3% w/w, for example, between 0.2% to 0.5%, 0.5% to 1%, 1% to 1.5%, 1.5% to 2%, or 2.5% to 3% w/w and the HA at a concentration of 0.2 mcg/mL-10 mcg/mL, for example between 0.2 to 0.5, 0.5-1.0, 1.0-1.5, 1.5-2, 2-3, 3-4, 4-5, 5-6, 6-7, 7-8, 8-9, or 9-10 mcg/mL. In one embodiment, the HA+P formulation is pre-loaded as a gel in an applicator. In one embodiment, the HA+P formulation in gel format in an applicator is used for to balance vaginal pH to it is desired 3.5-4.5 range in addition to the treatment of vaginal discomfort, for example for vaginal hydration to treat dryness.

In one embodiment, the gel formulation disclosed herein comprises both pectin (P) and collagen (Cg) as the active ingredients, with the pectin at a concentration of 0.2%-3% w/w, for example, between 0.2% to 0.5%, 0.5% to 1%, 1% to 1.5%, 1.5% to 2%, or 2.5% to 3% w/w and the Cg at a concentration of 5 mcg/mL-150 mcg/mL, for example between 5-10, 10-20, 20-30, 30-40, 40-50, 50-60, 60-70, 70-80, 80-90, 90-100, 100-110, 110-120, 120-130, 130-140, or 140-150 mcg/mL. In one embodiment, the Cg+P formulation is pre-loaded as a gel in an applicator. In one embodiment, the Cg+P formulation in gel format in an applicator is used for to balance vaginal pH to it is desired 3.5-4.5 range in addition to the treatment of vaginal discomfort such as vaginal tissue repair.

In one embodiment, the gel formulation disclosed herein comprises pectin (P), hyaluronic acid (HA), and collagen (Cg) as the active ingredients, with the pectin at a concentration of 0.2%-3% w/w, for example, between 0.2% to 0.5%, 0.5% to 1%, 1% to 1.5%, 1.5% to 2%, or 2.5% to 3% w/w, the HA at a concentration of 0.2 mcg/mL-10 mcg/mL, for example between 0.2 to 0.5, 0.5-1.0, 1.0-1.5, 1.5-2, 2-3, 3-4, 4-5, 5-6, 6-7, 7-8, 8-9, or 9-10 mcg/mL, and the Cg at a concentration of 5 mcg/mL-150 mcg/mL, for example between 5-10, 10-20, 20-30, 30-40, 40-50, 50-60, 60-70, 70-80, 80-90, 90-100, 100-110, 110-120, 120-130, 130-140, or 140-150 mcg/mL. In one embodiment, the Cg+P+HA formulation is pre-loaded as a gel in an applicator. In one embodiment, the Cg+P+HA formulation in gel format in an applicator is used for to balance vaginal pH to it is desired 3.5-4.5 range, for vaginal hydration, and for the treatment of vaginal discomfort such as vaginal tissue repair.

### (f). Foams

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants in general are not hydrocarbon propellant gases, which can produce flammable or explosive vapors during spraying. Furthermore, the compositions disclosed herein contain no volatile alcohols, which can produce flammable or explosive vapors during use.

### (g). Panty Liners and Absorbable Articles

In some embodiments, at least one active ingredient may be in substantially dry or solid form, such as a powder attached to or disposed within a pad. When wetted by water or aqueous fluids prior to, during, or after application of the active ingredient to the body, the active ingredient may at least partially dissolve to more effectively control the acidity of the environment (e.g., that of material on the skin of the pudendum) or to deliver other benefits. A wide variety of absorbent articles may be used to deliver formulations or assist in modifying the conditions of the pudendum to inhibit odor formation and release. Absorbent articles can include feminine pads, interlabial devices, tampons, incontinence devices such as diapers and related articles, briefs, panties, and the like. Description of example products can be found in, by way of example only, the following: U.S. Pat. Nos. 7,201,743, 6,454,751, 5,830,206, 5,620,432, 6,620,146, 6,375,646, 3,905,372; 2,662,527, 4,631,062. See also, US 2015/50150792 which describes produce and methods for use on the pudendum, including absorbent articles.

### (h). Vaginal Wipes

Methods of incorporating active agents of choice onto a vaginal wipe are known and have been described. The wipes can include skin-soothing Vitamins E, A & D. Cleaning compositions and their use in feminine hygiene wipes are described for example in U.S. Pat. No. 6,844,303 (incorporated herein by reference).

Briefly, the sheet of absorbent or porous material for use in the products of this invention can take the form of a tissue, a wipe, towel, towelette, and the like. The material may be flushable. As used herein, by “flushable” is meant that the material will pass through at least 10 feet of waste pipe in two toilet flushes. The material may also be biodegradable.

Sheet materials that can be used can be mono or multi-layered, woven or non-woven. They can be made of one or of several materials. For example, non-woven materials that have a web structure of fibrous or filamentous nature, in which the fibers or filaments are distributed randomly or with a certain degree of orientation, the former being obtainable by air-laying or by certain wet-laying processes, the latter by other wet-laying or by carding processes. The fibers or filaments can be natural, for example wood pulp, wool, cotton, linen and the like, or synthetic, for example polyvinyls, polyesters, polyolefins, polyamides and the like.

The wipe may be provided with the composition already present, such as a wet wipe or impregnated wipe holding the viscous carrier and active ingredients. Alternatively, the composition may be provided separately for the user to apply using a wipe or other substrate such as a tissue. In one version, a single-use pouch or kit comprises a wipe and a separate dose of the composition that can be released on to the wipe prior to application.

Wipes such as described in U.S. Pat. No. 6,844,303 can be modified to incorporate an effective amount of active agents disclosed herein. Vaginal wipes and wipe containers are described for example, in US 2015/0150792.

In one embodiment, the wipes formulation disclosed herein comprises pectin as the only active ingredient at a concentration of 0.2%-3% w/w, for example, between 0.2% to 0.5%, 0.5% to 1%, 1% to 1.5%, 1.5% to 2%, or 2.5% to 3% w/w.

In one embodiment, the wipes formulation disclosed herein comprises both pectin (P) and hyaluronic acid (HA) as the active ingredients, with the pectin at a concentration of 0.2%-3% w/w, for example, between 0.2% to 0.5%, 0.5% to 1%, 1% to 1.5%, 1.5% to 2%, or 2.5% to 3% w/w and the HA at a concentration of 0.2 mcg/mL-10 mcg/mL, for example between 0.2 to 0.5, 0.5-1.0, 1.0-1.5, 1.5-2, 2-3, 3-4, 4-5, 5-6, 6-7, 7-8, 8-9, or 9-10 mcg/mL.

### (i). Nano- and Microparticles

The one or more compounds, and optional one or more additional active agents, can be incorporated into microparticles, nanoparticles, or combinations thereof that provide controlled release of the compounds and/or one or more additional active agents. In embodiments wherein the formulations contain two or more drugs, the drugs can be formulated for the same type of controlled release (e.g., delayed, extended, immediate, or pulsatile) or the drugs can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading at the site in need of treatment, by means including enzymatic degradation, surfactant action of bile acids, and/or mechanical erosion. As used herein, the term “slowly soluble in water” refers to materials that are not dissolved in water within a period of 30 minutes. Examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and triglycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl cellulose, hydroxypropyl cellulose, methyl cellulose, and carboxymethylcellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

### Method of Making Nano- and Microparticles

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In some processes, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water-soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### (j). Injectable/Implantable Formulations

The compounds described herein can be incorporated into injectable/implantable solid or semi-solid implants, such as polymeric implants, solutions, suspensions and gels. In one embodiment, the compounds are incorporated into a polymer that is a liquid or paste at room temperature, but upon contact with aqueous medium, such as physiological fluids, exhibits an increase in viscosity to form a semi-solid or solid material. Example polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

### (k). Solid Dosage Formulations

Suitable solid dosage forms include suppositories, tablets, and capsules. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Some of the suppository formulations are designed to melt in vaginal cavity and release the active constituent over prolong period of time. Although suppository systems are most commonly used to administer drugs like dehydroepiandrosterone sulphate for ripening effect on uterine cervix, miconazole for vaginal candiasis and progesterone for hormonal replacement therapy, similar tablet and suppository formulations can be adapted to be used for the delivery of the active ingredients disclosed herein as well. Normal vaginal tablets contain similar components as like conventional oral tablets, they are easy to manufacture and insert. Mucoadhesive polymers are sometimes used in vaginal tablet formulation to increase the vaginal residence time. The polystyrene sulfonate (PSS) for example shows superior antimicrobial activity against HIV and HSV, therefore it is formulated in the form of vaginal tablet, which will not immobilize sperm, not cytotoxic and did not inhibit normal vaginal flora, so as proved as potential delivery system as discussed for example in Alam A. M., Ahmad J. F., Khan I. Z., Khar K. R. & Ali M., Development and evaluation of acid buffering bio adhesive vaginal tablet for mixed vaginal infections. AAPS Pharm Sci. Tech., 2007, 8(4): E1-E8. The presence of hydrophobic and release retarding materials may decrease the absorption of a drug from a vaginal formulation and too hydrophobic drugs may not be suitable for vaginal tablets. Further presence of penetration enhancers such as surfactants, bile salts can significantly enhance absorption.

In one embodiment, the suppository formulation disclosed herein comprises both pectin (P) and collagen (Cg) as the active ingredients, with the pectin at a concentration of 0.2%-3% w/w, for example, between 0.2% to 0.5%, 0.5% to 1%, 1% to 1.5%, 1.5% to 2%, or 2.5% to 3% w/w and the Cg at a concentration of 5 mcg/mL-150 mcg/mL, for example between 5-10, 10-20, 20-30, 30-40, 40-50, 50-60, 60-70, 70-80, 80-90, 90-100, 100-110, 110-120, 120-130, 130-140, or 140-150 mcg/mL. In one embodiment, the Cg+P formulation is pre-loaded as a suppository in an applicator. In one embodiment, the Cg+P formulation in suppository format in an applicator is used to balance vaginal pH to it is desired 3.5-4.5 range in addition to the treatment of vaginal discomfort such as vaginal tissue repair.

In one embodiment, the suppository formulation disclosed herein comprises pectin (P), hyaluronic acid (HA), and collagen (Cg) as the active ingredients, with the pectin at a concentration of 3 mcg/mL to 80 mcg/mL, for example, between 3-5, 5-10, 10-15, 15-20, 20-25, 25-30, 30-40, 40-50, 50-60, 60-70, and 70-80 mcg/mL, the HA at a concentration of 0.2 mcg/mL-10 mcg/mL, for example between 0.2 to 0.5, 0.5-1.0, 1.0-1.5, 1.5-2, 2-3, 3-4, 4-5, 5-6, 6-7, 7-8, 8-9, or 9-10 mcg/mL, and the Cg at a concentration of 5 mcg/mL-150 mcg/mL, for example between 5-10, 10-20, 20-30, 30-40, 40-50, 50-60, 60-70, 70-80, 80-90, 90-100, 100-110, 110-120, 120-130, 130-140, or 140-150 mcg/mL. In one embodiment, the Cg+P+HA formulation is pre-loaded as a suppository in an applicator. In one embodiment, the Cg+P+HA formulation in suppository format in an applicator is used for to balance vaginal pH to it is desired 3.5-4.5 range, for vaginal hydration, and for the treatment of vaginal discomfort such as vaginal tissue repair.

Formulations may be prepared using a pharmaceutically acceptable carrier. As generally used herein “carrier” includes, but is not limited to, diluents, preservatives, binders, lubricants, disintegrators, swelling agents, emollients, fillers, stabilizers, and combinations thereof.

Carrier also includes all components of the coating composition, which may include plasticizers, pigments, colorants, penetration enhancers, stabilizing agents, and glidants.

“Preservatives” can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethyl cellulose, hydroxypropyl cellulose, ethyl cellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginate, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Penetration enhancers” are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols). Examples of these classes are known in the art.

Forms, such as capsules, tablets, solutions, and suspensions, can be formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium. The particles can be formed of the drug and a controlled release polymer or matrix. Alternatively, the drug particles can be coated with one or more controlled release coatings prior to incorporation into the finished dosage form.

### Extended-Release Dosage Forms

Extended-release formulations are generally prepared as diffusion or osmotic systems, for example, as described in “Remington—The science and practice of pharmacy” (20th ed., Lippincott Williams & Wilkins, Baltimore, MD, 2000). The extended-release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkyl celluloses such as hydroxypropyl-cellulose, hydroxypropylmethyl cellulose, sodium carboxymethylcellulose, and Carbopol® 934, polyethylene oxides and mixtures thereof. Fatty compounds include, but are not limited to, various waxes such as carnauba wax and glyceryl tri stearate and wax-type substances including hydrogenated castor oil or hydrogenated vegetable oil, or mixtures thereof.

In certain embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain embodiments, the acrylic polymer is comprised of one or more amino methacrylate copolymers. Amino methacrylate copolymers are well known in the art, and are described in NF XVII as fully polymerized copolymers of acrylic and methacrylic acid esters with a low content of quaternary ammonium groups.

In one embodiment, the acrylic polymer is an acrylic resin lacquer such as that which is commercially available from Rohm Pharma under the tradename EUDRAGIT t®. In further embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also used. The code designations RL (high permeability) and RS (low permeability) refer to the permeability properties of these agents. EUDRAGIT® RL/RS mixtures are insoluble in water and in digestive fluids. However, multi particulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained release multi particulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended-release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended-release system by means of either applying an immediate release layer on top of the extended-release core using a coating or compression process or in a multiple unit system such as a capsule containing extended and immediate release beads.

Extended-release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended-release tablets containing wax materials are generally prepared using methods known in the art such as a direct blend method, a congealing method, and an aqueous dispersion method. In the congealing method, the drug is mixed with a wax material and either spray-congealed or congealed and screened and processed.

### Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Coating materials include bioerodible, gradually hydrolyzable, gradually water-soluble, and/or enzymatically degradable polymers. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, for example formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. In some embodiments, a stabilizing agent is used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxypropyl cellulose, hypromellose, lanolin, hydrous, lanolin alcohols, lecithin, medium-chain triglycerides, methylcellulose, mineral oil and lanolin alcohols, monobasic sodium phosphate, mono ethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

### Method of Use

The disclosed compositions and formulations are administered to a subject in need thereof, to ameliorate one or more symptoms associated with genitourinary syndrome of menopause (including vaginal dryness/atrophy), urinary tract infections, wound healing, skin conditions etc. In some forms, the formulation is administered topically to the vaginal pudendum. In some forms the formulation is administered intravaginally, in the form of a suppository, cream, gel etc., optionally, with a vaginal applicator.

Vaginal dryness may occur in patients with menopause, on chemotherapy, diabetes, etc.

Vaginal atrophy, one of the syndromes grouped unto genitourinary syndrome of menopause (which also includes vulvovaginal atrophy [VVA], urogenital atrophy, or atrophic vaginitis) is most commonly due to decreased estrogen levels (up to 95% reduction), particularly in postmenopausal women or women of any age who experience a decrease in estrogenic stimulation of the urogenital tissues resulting in changes to the anatomy and physiology of the genitourinary system. This results in a myriad of symptoms characteristic of VVA, such as dryness, burning, itching, vaginal discomfort, pain and burning when urinating, dyspareunia (painful sex), and spotting during intercourse.

In the first few years after the termination of menstruation, studies indicate that about 4% of women have clinical manifestations of VA early in menopause. However, as the decline in estrogen levels increase, dystrophic and atrophic changes develop in the vaginal mucosa, vulva, and other structures of the urogenital tract occur and this is observed in most women as time progresses ultimately resulting in a lower quality of life in such women compared to asymptomatic postmenopausal women.

In premenopausal women however, triggers of vaginal dryness include but are not limited to breastfeeding, intensive vaginal douching, cigarette smoking, autoimmune conditions such as Sjogren's syndrome, pathologic conditions such as diabetes, premature ovarian insufficiency (e.g., due to premature aging), bilateral oophorectomy, ovarian failure due to radiation or arterial embolization, hypothalamic-pituitary disorders. as well as medication classes such as antidepressants, antiestrogenic (e.g., to treat endometriosis and uterine fibroids), and anticholinergic medications. Also at risk are breast cancer survivors suffering from consequences from treatment such as chemotherapy or aromatase inhibitors.

The method includes administering the compositions described herein, to a subject in need thereof. The method in some embodiments providing one or more wipes for use with the product, and the step of providing directions to the user may then include providing directions to transfer the product from at least one of the one or more wipes to the pudendum.

### Examples

### Materials and Methods

The active ingredient collagen (Cg) used herein is from marine source purchased from Strauber (https://www.stauberusa.com/), having MW of about 5 kDa. The active ingredient hyaluronic acid (HA) used herein is purchased from Bloomage Biotech (https://www.bloomagebioactive.com/En), having a MW of about 0.3 MDa. The active ingredient pectin used here is a high methoxylated pectin sourced from citrus peel, having a MW of 80-110 KDa.

### Formulations

Formulations used in the examples are outlined in Table 2 below. Aliquots of the formulations from table one is used to produce the final testing solutions added into the hVECs, for example, in the final concentrations of 0.5, 1.5, and 5 mg/mL.

TABLE 2

| | Formulation (% w/w) | | | | | | |
|---|---|---|---|---|---|---|---|
| | A | B | C | D | E | H | I |
| Ingredients | P | HA + P + Cg | Cg + P | HA + P | Cg | HA | HA + Cg |
| HA | 0 | 0.2 | 0 | 0.2 | 0 | 0.2 | 0.2 |
| P | 2 | 2 | 2 | 2 | 0 | 0 | 0 |
| Cg | 0 | 10 | 10 | 0 | 10 | 0 | 10 |
| Glycerin | 10 | 10 | 10 | 10 | 10 | 10 | 10 |
| Lactic Acid | 0.1 | 0.3 | 0.5 | 0.3 | 0.6 | 0.4 | 0.3 |
| Phenoxyethanol | 0.5 | 0.5 | 0.5 | 0.5 | 0.5 | 0.5 | 0.5 |
| Water | 87 | 86.9 | 77.0 | 87 | 78.9 | 88.9 | 79 |
| Consistency | fluid | gel | viscous | fluid | thin | thin | fluid |
| | gel | | liquid | gel | fluid | fluid | gel |
| pH | 3.7 | 4.0 | 4.1 | 4.0 | 4.1 | 4.05 | 4.1 |

HA = hyaluronic acid,
P = pectin,
Cg = Active ingredient collagen

In general, a 1% solution of Pectin used herein, between pH 3 and 3.7 is in a gel format, and outside of this range, it's a thin flowable fluid.

The effect of pectin at different concentrations on the effect of healthy vaginal bacteria is studied and the results shown in FIG. 2. Specifically, pectin was introduced into broth of 3 different bacteria, L. crispatus, L. gasseri, L. jensenii known to be beneficial and prevalent in healthy the vaginal microbiome to evaluate potentiation of growth which would lead to increased production of lactic acid. The reduction in pH due to lactic acid production after 24 hours was measured. The decrease in pH shown in FIG. 2 indicates pectin is beneficial as a buffer and as a prebiotic to feed to beneficial bacteria, in turn dropping and maintaining the pH at the beneficial range of 3.5 to 4.5. Formulation containing pectin disclosed herein therefore can be used to bring vaginal pH of a subject to the beneficial range of 3.5 to 4.5.

When pectin combines with either HA, or Cg, or Cg+HA (as a trio), it tends to reduce the pH to a degree relative to the combination, even before adding the pH adjuster such as lactic acid, as shown in Table 3 below.

TABLE 3

| | Initial pH | pH post- |
|---|---|---|
| | (before addition | addition |
| | of prebiotic | of prebiotic |
| Combination | pectin) | (pectin) |
| A | 3.6 | 3.6 |
| B | 6.5 | 5.2 |
| C | 6.5 | 5.2 |
| D | 6.1 | 4.3 |
| E | 6.5 | |
| H | 6.1 | |
| I | 5.9 | |

Collagen alone remains a thin, soluble solution at pH above 5.5. Once pH is decreased with addition of lactic acid, thickening was observed. HA on the other hand is known to degrade at pHs<4 and >11. Collagen gives higher viscosity but hydrolyzed version gives low viscosity regardless of concentration.

Given that the end product of estrogen repletion is collagen synthesis in the vaginal wall, the disclosed formulation is targeted towards markers of collagen synthesis. The following experiments were carried out in order to determine ability of test compositions to increase collagen and impact vaginal ultrastructure.

### Optimization of Human Vaginal Epithelial Cell and Collagen Release Assay Investigational Product

Lifeline Technology's Human Vaginal Epithelial Cells (hVECs) 2-dimensional growth format were utilized as an initial relevant human cell platform for evaluating the mRNA expression levels of targets and secreted procollagen type 1 c-peptide (PIP) for all tested formulations. This assay is optimized for detection of very low levels of collagen in lysates.

hVECs were grown in 96-well format with 7,500 cells/well. Lysates from hVECs were prepared by discarding media, adding 100 μL RIPA buffer with 1× protease inhibitor, incubation for 30 minutes on ice, followed by centrifugation for 10 minutes at 12,000 RPM. Cell lysates were diluted 1:10 with ddH₂O to achieve a detectable fluorescence signal within the standard curve generated using reference collagen as per manufacturer's protocol (Fluorometric Sigma collagen assay kit).

Because all concentrations of the different compositions (as shown in Table 1) tested indicated cytotoxic properties past 10 mg/mL, collagen release assays using Sigma Aldrich Collagen Assay Kit (Catalog Number MAK322, following the manufacturer's directions) were performed at concentrations ≤5 mg/mL for each formulation to ensure cell viability throughout the duration of the experiment.

2% Hyaluronic Acid (HA) was used as control. Further, this concentration is within range of publications associated with the product (Sigma 53163).

### Gene Expression Assay

A subsequent experiment incubating hVECs with P only, HA+P+Cg, P+Cg, HA+P at the optimal concentrations and time periods established above (vs. positive and negative ctr) was carried out.

For cell studies, cells were washed, lysed, and RNA was extracted for gene expression studies. Downstream TaqMan qPCR was performed on cDNA synthesized from freshly isolated RNA. N=4 for each treatment group. All biological samples were stored at −80° C.

TaqMAN Gene Expression Analysis, an industry gold-standard assay for detecting mRNA transcript level was used to detect expression of the following key targets:

TaqMan Gene Targets

COL1A1- Collagen 1
COL3A1- Collagen 3
MMP1- Matrix metalloproteinase 1
MMP3- Matrix metalloproteinase 3
TIMP1-Tissue inhibitor of metalloproteinases
BTC-Betacellulin

Collagen Assay: Catalog Number MAK322. Experiments were conducted according to manufacturer's protocol.

### Results

Identification of Best Parameters for Applying Sigma Collagen Assay Kit to hVEC Lysates

This assay is optimized for detection of very low levels of collagen in lysates. hVECs were grown in 96-well format with 7,500 cells/well. Lysates from hVECs were prepared by discarding media, adding 100 μL RIPA buffer with 1× protease inhibitor, incubation for 30 minutes on ice, followed by centrifugation for 10 minutes at 12,000 RPM. Lysates were diluted 1:10 with ddH2O to achieve a detectable fluorescence signal within the standard curve generated using reference collagen as per protocol.

### Collagen Release Assay

To assess the effect of test formulations on inducing collagen production in hVECs, we used a collagen assay kit to quantify collagen in cell lysates via a fluorimetric assay. The fluorescence intensity measured was in direct correlation with the collagen concentration in the sample.

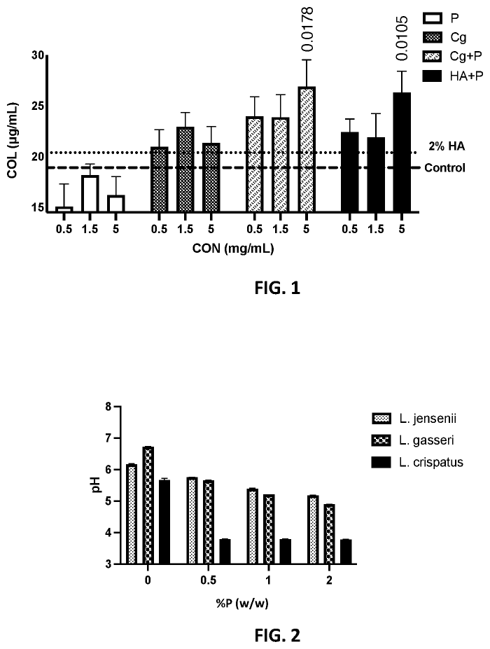

Collagen is detectable in cell lysates using the Sigma Collagen Assay kit conditions (Catalog Number MAK322). The effects of the formulations disclosed herein on collagen synthesis is summarized in FIG. 1. As shown in FIG. 1, the treatment of cells with 2% hyaluronic acid control shown as a dotted line, right above the control, which is shown as a dashed line, indicated that HA alone did not significantly stimulate collagen production in hVECs compared with untreated controls. However, as shown in FIG. 1, Cg+P and HA+P treatment resulted in significant increased levels of collagen in treated cells compared with untreated controls. In another words, as shown in FIG. 1, in a dose dependent manner, both actives (HA and Cg) perform better in the presence of pectin.

### Gene Expression Data

### HA+P Vs HA Only

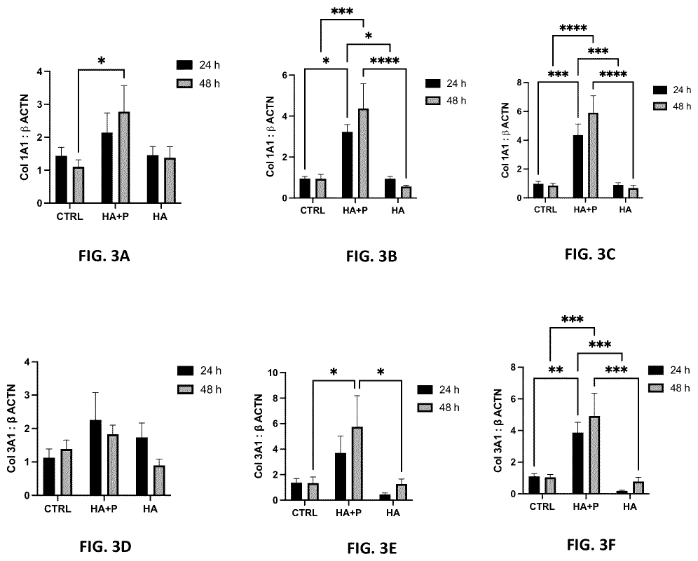

Experiments were carried out to determine whether treatment of hVECs with formulations containing either HA or HA+ Pectin would result in increased collagen gene expression in a dose and time dependent manner. Specifically, gene expression studies were conducted to ascertain whether there is a time and/or dose-dependent effect observed in mRNA expression of Col1A1 and Col3A1 after treatment with varying concentrations of formulation HA+P or HA alone. The data is shown in FIGS. 3A-3F. FIGS. 3A-3C are representative graphs showing a dose dependent increase in gene expression of Col1A1 after treatment with formulations HA+P and HA only. At each concentration, the treatment with HA+P formulation either showed a trend towards increase or resulted in significant increase in Col1A1 gene expression compared to either the untreated controls or with the HA only formulation. FIGS. 3D-3E show the effect of HA+P on Col3A1 gene expression in hVECs. Treatment of hVECs with HA+P resulted in significant increase in Col3A1 gene expression levels especially at 1.5 mg/mL and 3 mg/mL concentrations compared to control and compared to HA only treatments.

Specifically, the data shows that at the 3 mg/mL concentration, there is a significant increase in Col1A1 gene expression in the HA+P treatment condition both at the 24 h and 48 h time points, compared to either control or to the HA only treated cells. The results show that that addition of Pectin to HA significantly increases Col1A1 gene expression levels by 4-6-fold. The effect of Pectin in potentiating the effect of HA on collagen gene expression demonstrates that addition of Pectin to HA is beneficial to hVECs ability to express collagen. Though previous studies have stated that HA induces collagen release in the skin, no study has demonstrated that addition of pectin to HA will result in such a dramatic increase in collagen release from hVECs, nor could such a dramatic increase in collagen gene expression have been predicted from the skin studies.

### Collagen+P vs Collagen Only

Experiments were carried out to determine whether treatment of hVECs with a formulation containing Collagen+Pectin would result in an increase Col1A1 and Col3A1 gene expression in a dose and time dependent manner. To investigate the effect of formulation Cg+P on gene expression of Col1A1 and Col3A1, hVECs were treated with different concentrations of formulation Cg+P and incubated for either 24 or 48 h.

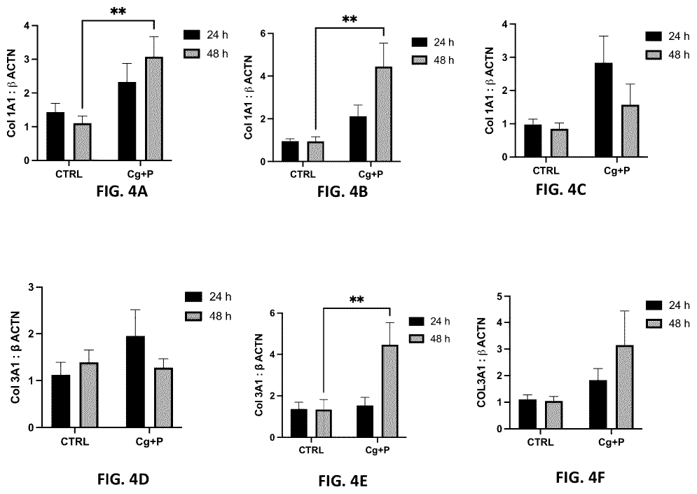

As shown in FIGS. 4A-4E, after 24 hours of treatment, there is a trend towards increased expression of Col1A1 in treated cells compared to untreated controls; however, after 48 h of incubation, cells treated with formulation Cg+P showed significant increase in Col1A1 at the 0.15 mg/ml and 1.5 mg/mL concentrations as well as increase in Col3A1 at the 1.5 mg/mL concentration.

These results show that treatment of hVECs with formulation compared to untreated controls; however, after 48h of incubation, cells treated with formulation Cg+P promote Col1A1 and Col3A1 gene expression and can therefore be beneficial in improving the vaginal wall ultrastructure given type 1 and type 3 collagens are major components of the vaginal epithelium and epithelial tissue (You et al 2020).

Collagen Only Vs. Control

Experiments were conducted to determine whether treatment of hVECs with a formulation containing Collagen would affect Col1A1 and Col3A1 gene expression in a dose and time dependent manner.

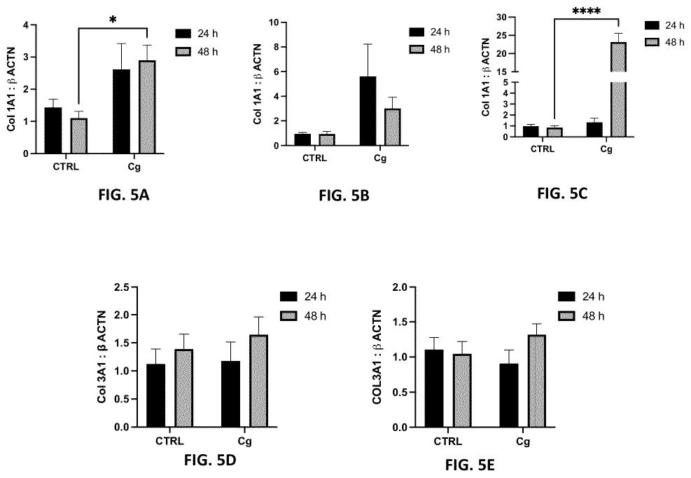

As shown in FIGS. 5A-5C, treatment of hVECs with formulation Collagen only or Cg showed significantly increased Col1A1 gene expression at the 48-hour time point at both concentrations—0.15 mg/mL and 3 mg/mL. For the 1.5 mg/mL concentration, a trend towards increased Col1A1 gene expression was observed in treated cells compared to controls though this was not significant. FIG. 5D-E shows that there was no significant increase in Col3A1 gene expression was observed in the treated cells compared with untreated controls. Thus, treatment of cells with collagen only formulation more specifically induce Col1A1 compared to Col3A1. Compared to the data shown in FIGS. 4A-4E, pectin improves collagen's ability to improve Col3A1 gene expression.

### Collagen Only Vs HA Only

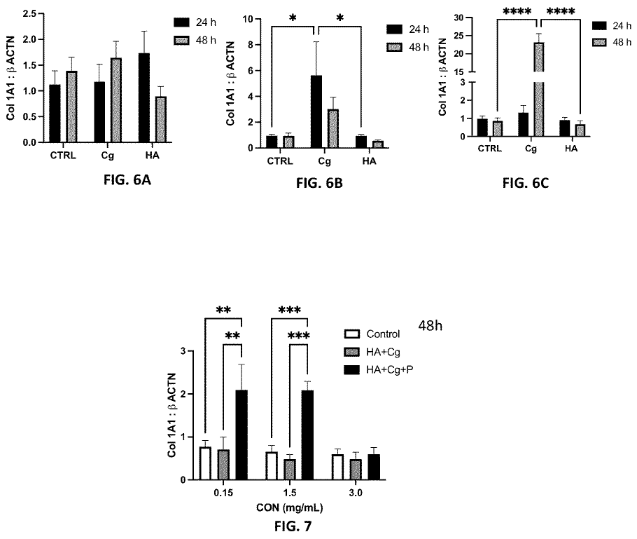

Experiments were conducted to determine which formulation (HA or Col) will induce a greater increase in Col1A1 gene expression in hVECs after incubation for 24h or 48 h. The data in FIGS. 6A-6C, show that at the lowest concentration 0.15 mg/mL, there was no difference observed between treated and untreated controls; however, at the higher concentrations (1.5 mg/mL and 3 mg/mL), cells treated with formulation Cg only showed a trend toward increase or significant increase in Col1A1 gene expression compared with both untreated controls as well as cells treated with formulation HA only. These results show that at these studied concentrations, Cg only formulation is able to induce Col1A1 gene expression in hVECs to a greater extent compared to HA only formulation.

### Collagen+Pectin+HA Vs Collagen+HA

Experiments were conducted to determine the effect of combination formulation Cg+HA in the presence or absence of P, on gene expression of Col1A1. hVECs were incubated for a period of 48h with varying concentrations of either HA+Cg or HA+Cg+P. The data in FIG. 7 show that in the absence of P, HA+Cg formulations do not stimulate Col1A1 gene expression. HA+Cg+P formulations however significantly induced gene expression of Col1A1 within a concentration range of 0.15-1.5 mg/ml compared to either untreated controls or HA+Cg formulations. These results show that at these studied concentrations, pectin promotes HA+Cg induced Col1A1 gene expression in hVECs to a greater extent compared to HA+Cg only formulation.

The function of various formulations disclosed herein are summarized in the table 4 below.

TABLE 4

| Active | |
|---|---|
| Ingredient Combination | Function |
| P only | pH-balancing (prebiotic) |
| P + Cg | pH-balancing (prebiotic) + Repair |
| P + HA | pH-balancing (Prebiotic) + hydration |
| Cg + HA + P | pH-balancing (Prebiotic) + |
| | repair + hydration |
| Cg | Repair only |
| HA | Hydration only |
| Cg + HA | Repair + hydration |

When the formulations were adjusted to a pH between 3.6-4 (with lactic acid) as captured in table below. Most of the formulations below were either thick gels or fluidic gels upon dropping the pH to 4, as detailed in the table 5 below.

TABLE 5

| | A | B | C | D | E | H | I |
|---|---|---|---|---|---|---|---|
| HA | 0% | 0.2% | 0% | 0.2% | 0% | 0.2% | 0.2% |
| Cg | 0% | 10% | 10% | 0% | 10% | 0% | 10% |
| P | 2% | 2% | 2% | 2% | 0% | 0% | 0% |

pH (adjustments w/lactic acid)

| | | | | | | | |
|---|---|---|---|---|---|---|---|
| innate/initial pH | 3.6 | 5.2 | 5.2 | 4.28 | 6.5 | 6.1 | 5.9 |
| adj pH | 3.9 | 4.05 | 4.01 | 3.7 | 3.6 | 4 | 5.9* |

Note:
Combinations containing Pct naturally drops in pH slightly due to pectin being a low pH solution. Combinations w/o pectin remained at ~>6.0 and needed to be adjusted further to ~4.0 pH with lactic acid. However, HA drops the pH naturally more than Cg does
% in table 5 is w/w %.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition for treating one or more symptoms associated with vaginal atrophy or discomfort, comprising,
an effective amount of an active agent pectin (P) with an optional second active agent selected from the group consisting of hyaluronic acid (HA), collagen (Cg), and a combination of HA and Cg, in a pharmaceutically acceptable carrier, for vaginal delivery,
wherein the composition has a pH between 3.5 and 4.5.

2. The composition of claim 1, comprising an effective amount of P only.

3. The composition of claim 1, comprising an effective amount of P in combination with HA.

4. The composition of claim 1, comprising an effective amount of P in combination with Cg.

5. The composition of claim 1, comprising an effective amount of P, HA, and Cg.

6. The composition of claim 1, wherein the pharmaceutically acceptable carrier comprises at least one of glycerin and phenoxyethanol.

7. The composition of claim 1, wherein the pharmaceutically acceptable carrier further comprising lactic acid.

8. The composition of claim 1, further comprising additives.

9. The composition of claim 1, further comprising additives to provide delayed release.

10. The composition of claim 1, having a consistency of a gel.

11. The composition of claim 1, having a consistency of a viscous liquid.

12. The composition of claim 1, comprising by w/w %, 0.1-30% pectin, 0-15% Cg, and 0-10% HA.

13. The composition of claim 1, comprising by w/w %, 0.1-30% glycerin, 0-5% lactic acid, 0-1% phenoxyethanol, and 9-90% water.

14. A formulation comprising the composition of claim 1, formulated into a gel.

15. A formulation comprising the composition of claim 1, formulated into a vaginal suppository.

16. The formulation of claim 12, wherein the formulation is encapsulated in a vaginal applicator.

17. A formulation comprising the composition of claim 1, infused into a sheet of absorbent or porous material to form a tissue, a wipe, a towel, a towelette, or the like.

18. A method of treating one or more symptoms associated with vaginal atrophy or discomfort, comprising administering the composition or formulation of claim 1, to a subject in need thereof.

19. The method of claim 18, wherein the one or more symptoms are selected from the group consisting of pH imbalance, vaginal dryness, burning, itching, vaginal discomfort, pain and burning when urinating, dyspareunia (painful sex), and spotting during intercourse.

20. The method of claim 18, wherein the subject is a postmenopausal women.
